(19) United States
(12) Patent Application Publication
(10) Pub. No.: US 2024/0277883 A1
(43) Pub. Date: Aug. 22, 2024

(54) REFILLABLE VOLATILE COMPOSITION DISPENSER

(71) Applicant: The Procter & Gamble Company, Cincinnati, OH (US)

(72) Inventors: Iris Bi LIU, Mason, OH (US); Yi HAN, Mason, OH (US); Timothy Roy NIJAKOWSKI, Mason, OH (US)

(21) Appl. No.: 18/582,778

(22) Filed: Feb. 21, 2024

Related U.S. Application Data
(60) Provisional application No. 63/447,425, filed on Feb. 22, 2023.

(51) Int. Cl. A61L 9/12

(52) U.S. Cl. CPC A61L 9/122; A61L 2209/111; A61L 2209/133

(57) ABSTRACT

A volatile composition dispenser includes a top portion and a base portion. The top portion includes one or more slots for receiving one or more volatile composition cartridges. The base portion includes a rupture member. When the top portion is engaged with the base portion and rotated at least 45 degrees, the rupture member engages and ruptures a rupturable substrate of one or more of the one or more volatile composition cartridges, thereby allowing a volatile composition to be dispensed.

### FIELD

The present disclosure is directed to a replenishable volatile composition dispenser, and, more particularly, is directed to a volatile composition dispenser having housing that is capable of receiving a volatile composition cartridge having a volatile composition.

### BACKGROUND

Volatile compositions dispensers of various configurations are known. Some volatile composition dispensers are configured for one-time use and are then disposed of or recycled after the volatile composition is depleted. Volatile composition dispensers may be of a configuration that requires activation to release the volatile composition from the volatile composition dispenser for the first time. Such a volatile composition dispenser may only be activated one time, such that it would not be possible to refill or replenish the volatile composition and reactivate the volatile composition dispenser. As such, it would be beneficial to provide a volatile composition dispenser that is replenishable.

A further challenge is to maintain a volatile composition or cartridge in its original state from manufacture with no leakage of composition while in storage on a shelf prior to purchase, while also enabling activation of the cartridge easily and effectively when the consumer is ready to use the device. Therefore, there exists a need for an apparatus for activating the described cartridge after it has been inserted into the device.

### SUMMARY

Various embodiments provide a volatile composition dispenser that is easily replenishable and with which refill systems are easily activated after being inserted into the device. A volatile composition dispenser may include a top portion and a base portion. The top portion may include one or more slots for receiving one or more volatile composition cartridges. The base portion may include a rupture member. When the top portion is engaged with the base portion and rotated, the rupture member engages each of the one or more volatile composition cartridges in order to rupture a rupturable substrate thereof. This engagement may activate each cartridge, allowing a volatile composition to be dispensed through the rupturable substrate and thereafter through an optional microporous membrane of each cartridge.

### BRIEF DESCRIPTION OF THE DRAWINGS

Many aspects of this disclosure may be better understood with reference to the following figures.

FIG. 1 is an example according to various embodiments illustrating an upper front perspective view of a volatile composition dispenser.

FIG. 2 is an example according to various embodiments illustrating a lower front perspective view of a volatile composition dispenser.

FIG. 3 is an example according to various embodiments illustrating a front view of a volatile composition dispenser.

FIG. 4 is an example according to various embodiments illustrating a rear view of a volatile composition dispenser.

FIG. 5 is an example according to various embodiments illustrating a left side view of a volatile composition dispenser.

FIG. 6 is an example according to various embodiments illustrating a right view of a volatile composition dispenser.

FIG. 7 is an example according to various embodiments illustrating a top view of a volatile composition dispenser.

FIG. 8 is an example according to various embodiments illustrating a bottom view of a volatile composition dispenser.

FIG. 9 is an example according to various embodiments illustrating an exploded upper front perspective view of a volatile composition dispenser.

FIG. 10 is an example according to various embodiments illustrating an exploded front view of a volatile composition dispenser.

FIG. 11 is an example according to various embodiments illustrating an exploded left side view of a volatile composition dispenser.

FIG. 12 is an example according to various embodiments illustrating a lower front perspective view of a refill system carrier.

FIG. 13 is an example according to various embodiments illustrating an upper back perspective view of a refill system carrier.

FIG. 14 is an example according to various embodiments illustrating upper front perspective view of a refill system carrier.

FIG. 15 is an example according to various embodiments illustrating upper front perspective view of a refill system carrier.

FIG. 16 is an example according to various embodiments illustrating a perspective view of a volatile composition cartridge.

FIG. 17 is an example according to various embodiments illustrating a cross-sectional side view of a volatile composition cartridge.

FIG. 18 is an example according to various embodiments illustrating a perspective view of a refill system carrier and a disengaged refill system.

FIG. 19 is an example according to various embodiments illustrating a perspective view of a refill system carrier and an engaged refill system.

FIG. 20 is an example according to various embodiments illustrating a perspective view of a rupture assembly.

FIG. 21 is an example according to various embodiments illustrating a top perspective view of a rupture assembly.

FIG. 22 is an example according to various embodiments illustrating a top view of a base portion of a volatile composition dispenser.

FIG. 23 is an example according to various embodiments illustrating a cross-sectional view along line X-Y of a base portion of a volatile composition dispenser.

FIG. 24 is an example according to various embodiments illustrating a top view of a housing of a volatile composition dispenser.

FIG. 25 is an example according to various embodiments illustrating a cross-sectional view along line X-Y of a housing of a volatile composition dispenser.

FIG. 26 is an example according to various embodiments illustrating a cross-sectional view along line A-B of a housing of a volatile composition dispenser.

FIG. 27 is an example according to various embodiments illustrating a cross-sectional view along line A-B of a volatile composition dispenser.

FIG. 28 is an example according to various embodiments illustrating a cross-sectional view along line X-Y of a volatile composition dispenser.

FIG. 29 is an example according to various embodiments illustrating a perspective view of a refill system being inserted into a refill system carrier.

FIG. 30 is an example according to various embodiments illustrating a refill system carrier being inserted into a base portion of a volatile composition dispenser.

FIG. 31 is an example according to various embodiments illustrating a refill system carrier being rotated within a base portion of a volatile composition dispenser to cause a rupture member to rupture a rupturable substrate thus activating the cartridges of the refill system.

FIG. 32 is an example according to various embodiments illustrating a refill system carrier being rotated within a base portion of a volatile composition dispenser to disengage the refill system carrier from the base portion.

FIG. 33 is an example according to various embodiments illustrating a refill system carrier being removed from a base portion of a volatile composition dispenser.

FIG. 34 is an example according to various embodiments illustrating a refill system being released from a refill system carrier.

FIG. 35 is an example according to various embodiments illustrating a perspective view of an upper part of a base portion of a volatile composition dispenser.

FIG. 36 is an example according to various embodiments illustrating a top perspective view of an upper part of a base portion of a volatile composition dispenser.

FIG. 37 is an example according to various embodiments illustrating a bottom perspective view of an upper part of a base portion of a volatile composition dispenser.

FIG. 38 is an example according to various embodiments illustrating a top perspective view of an electrical support assembly of a volatile composition dispenser.

FIG. 39 is an example according to various embodiments illustrating a perspective view of an electrical support assembly of a volatile composition dispenser.

FIG. 40 is an example according to various embodiments illustrating an upper front perspective view of a lower part of a base portion of a volatile composition dispenser.

FIG. 41 is an example according to various embodiments illustrating a bottom perspective view of a lower part of a base portion of a volatile composition dispenser.

FIG. 42 is an example according to various embodiments illustrating an upper perspective view of a pedestal of a base portion of a volatile composition dispenser.

FIG. 43 is an example according to various embodiments illustrating a lower perspective view of a pedestal of a base portion of a volatile composition dispenser.

FIG. 44 is an example according to various embodiments illustrating a schematic diagram of various components of a volatile composition dispenser, an airflow therethrough, and an environment thereof.

It should be understood that the various embodiments are not limited to the examples illustrated in the figures.

### DETAILED DESCRIPTION

Various embodiments will now be described to provide an overall understanding of the principles of the structure, function, manufacture, and use of the apparatuses and methods disclosed herein. One or more examples of these embodiments are illustrated in the accompanying drawings. Those of ordinary skill in the art will understand that the apparatuses and methods specifically described herein and illustrated in the accompanying drawings are non-limiting example embodiments and that the scope of the various embodiments of the present disclosure is defined solely by the claims. The features illustrated or described in connection with one example embodiment may be combined with the features of other example embodiments. Such modifications and variations are intended to be included within the scope of the present disclosure.

Various embodiments relate to a fan assisted volatile composition dispenser or air freshener device and, more particularly, to activation of a refill system comprising one or more cartridges by rotation around a three-dimensional spindle, whereby during the rotation step, the shape and orientation of the three dimensional spindle applies pressure to a rupturable film or membrane within each cartridge, thereby releasing a volatile composition. In one embodiment, one or more motion activated sensors in the device activate the fan, passing air over the cartridges and evaporating the volatile composition from a microporous membrane on the other surface of the cartridge.

The volatile composition dispenser may also be configured as a non-energized volatile composition dispenser for the delivery of a volatile composition in a continuous manner. “Non-energized” can mean that the apparatus is passive and does not require to be powered by a source of external energy. The volatile composition dispenser does not need to be powered by a source of heat, gas, or electrical current, and the volatile composition is generally not delivered by aerosol means. The volatile composition dispenser may be configured as an unenergized system.

A volatile composition dispenser may be used to dispense at least one volatile composition and/or other solution or composition, such as a perfume, a fragrance, and/or an insecticide, for example, to an area or atmosphere surrounding the volatile composition dispenser. The volatile composition can comprise a single chemical or a single material that is capable of entering the vapor phase under atmospheric conditions or, more commonly, the volatile composition can comprise a mixture of chemicals and/or materials that are capable of entering the vapor phase under atmospheric conditions. The volatile composition can comprise, but is not limited to, a substance that can function as an air freshener, a deodorant, an odor neutralizing material, an odor blocking material, a malodor counteractant, an odor masking material, an aromatherapy material, an aromachology material, an insecticide, air and/or surface sanitizer, and/or a combination thereof. In other various embodiments, the volatile composition can comprise other various materials that can act in their vapor phase to modify, enhance, and/or treat an atmosphere or an area outside of the volatile composition dispenser.

The volatile composition dispenser may be configured to be used within an interior or outdoor space, e.g., a bathroom, a kitchen, a patio, or a vehicle, although the present disclosure is not limited to such use. Those of skill in the art will understand that the dispenser may be configured for use in any environment, such as a home or an office or a vehicle, and may be configured to dispense any suitable solution, chemical, material, and/or composition.

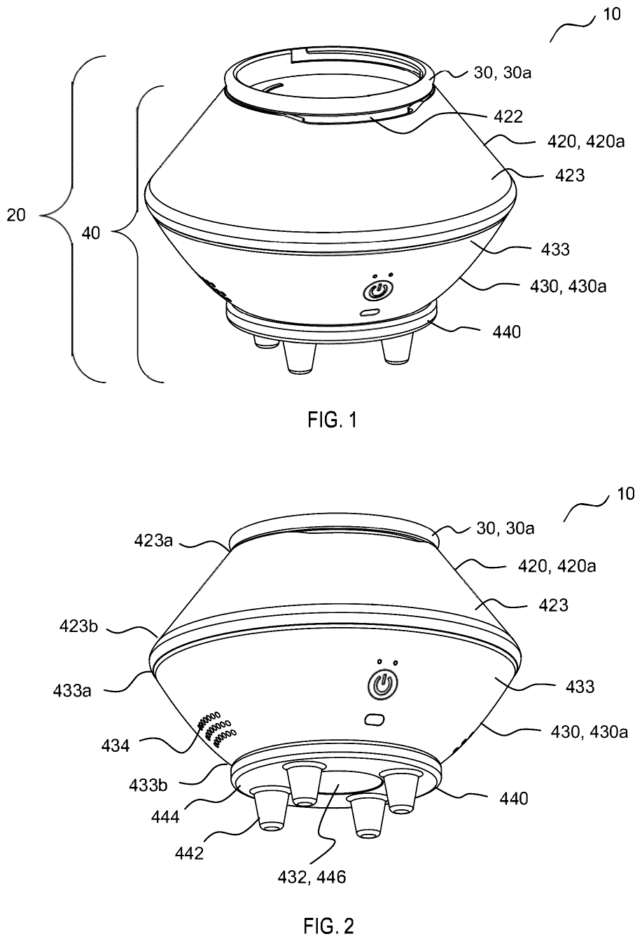

FIG. 1 is an example according to various embodiments illustrating an upper front perspective view of a volatile composition dispenser 10. The volatile composition dispenser 10 may comprise a housing 20. The housing 20 may comprise a top portion 30, which may be, for example, a refill system carrier 30a. The housing 20 may further comprise a base portion 40.

The base portion 40 may comprise an upper part 420, which may be, for example, an upper frustoconical cover 420a. The base portion 40 may further comprise a lower part 430, which may be, for example, a lower frustoconical cover 430a. Finally, the base portion 40 may comprise a pedestal 440. The upper part 420 may rest upon or engage the lower part 430 and the lower part 430 may rest upon or engage the pedestal 440. The top portion 30 may rest upon, be inserted into, or engage with the housing 20.

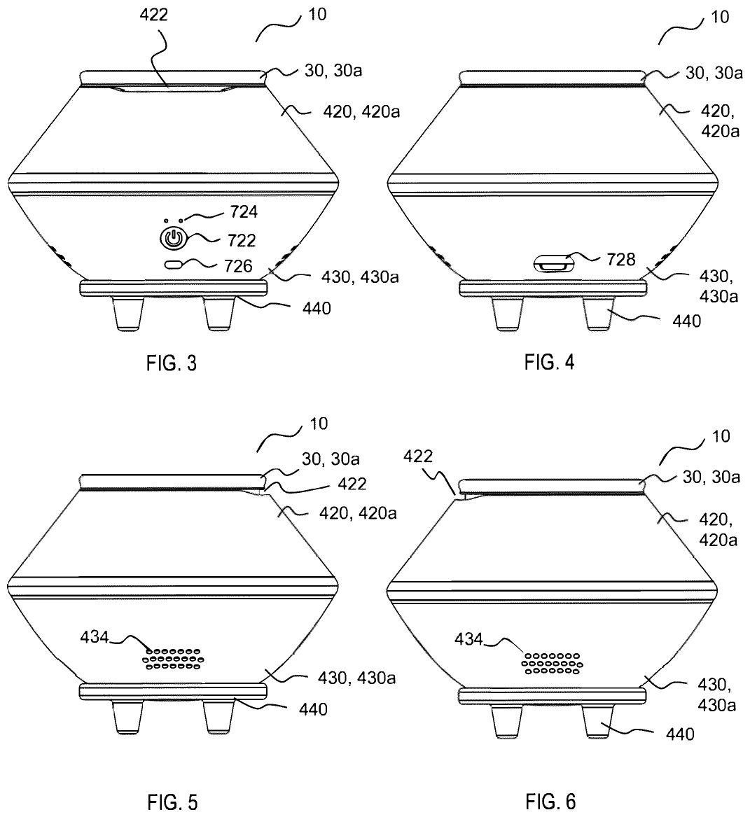

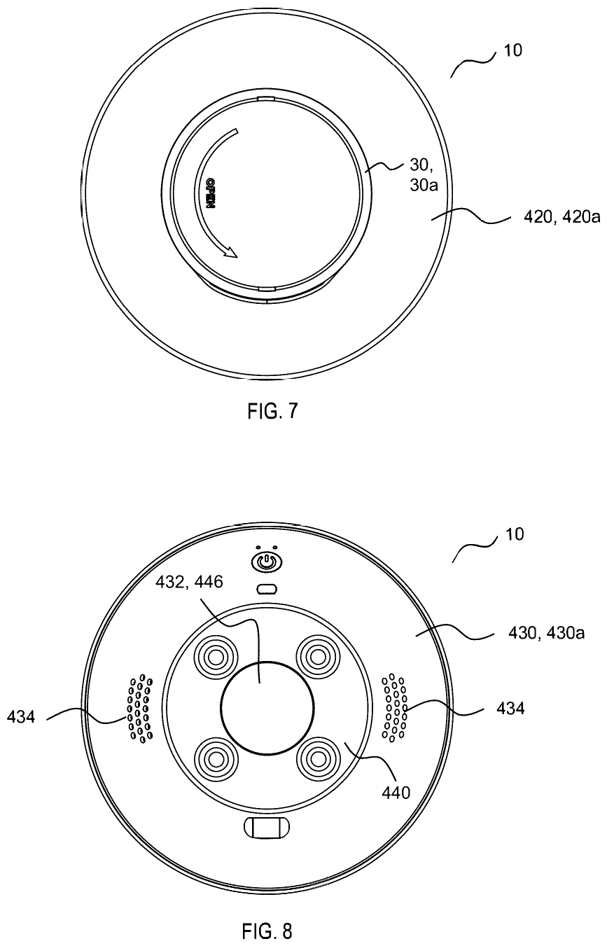

FIG. 2 provides a lower front perspective view, FIG. 3 provides a front view, FIG. 4 provides a rear view, FIG. 5 provides a left side view, FIG. 6 provides a right view, FIG. 7 provides a top view, and FIG. 8 provides a bottom view of a volatile composition dispenser 10. of a volatile composition dispenser 10. Referring to FIGS. 1-8, the upper part 420 may comprise one or more sidewalls 423. For example, the upper frustoconical cover 420a may comprise an upwardly inclined sidewall 423, that is wider at its base 423b than at its top 423a. Similarly, the lower part 430 may comprise one or more sidewalls 433. For example, the lower frustoconical cover 430a may comprise a downwardly inclined sidewall 433, that is wider at its top 433a than at its base 433b. Various features may be defined by or in the sidewalls 423, 433. For example, an air outlet 422 may be defined in or by the sidewall 423 of the upper part 420 of the base portion 40. As will be discussed hereinafter, air flow carrying a volatile composition may be directed through the air outlet 422. As best illustrated in FIG. 3, a power button 722, a power indicator light 724, and/or a motion detector 726 may be defined by or in the sidewall 433 of the lower part 430 of the base portion 40. The motion detector 726 may be configured to activate the fan 710 when motion is detected to release more volatile composition. The fan 710 may be provided with a logic circuit comprising a processor and a memory configured to provide a sleep mode, which may be configured to turn the fan 710 off and allow the device 10 to sleep for a time period of about 5 minutes or longer after a number of activations, such as, for example 3 to 6 activations. The fan 710 may be disposed at any suitable location within the housing 20, including in the base portion 40, or in the top portion 30. The volatile composition dispenser 10 may include additional mechanisms beyond the motion sensor 726, to switch on the fan 712, including accelerometers, acoustic, infrared, light, temperature, and vibration sensors. As best illustrated in FIG. 4, a power cord inlet 728 for a power cord (not shown) may be defined by or in the sidewall 433 of the lower part 430 of the base portion 40. Finally, as best illustrated in FIGS. 5, 6, and 8, one or more side air inlets 434 may be defined by or in the sidewall 433 of the lower part 430 of the base portion 40. These side air inlets 434 may allow air to flow into the base portion 40 to be mixed with a volatile composition and ultimately ejected via the air outlet 422. The pedestal 440 may comprise an aperture 446, which may allow illumination by an optional light through a translucent lens.

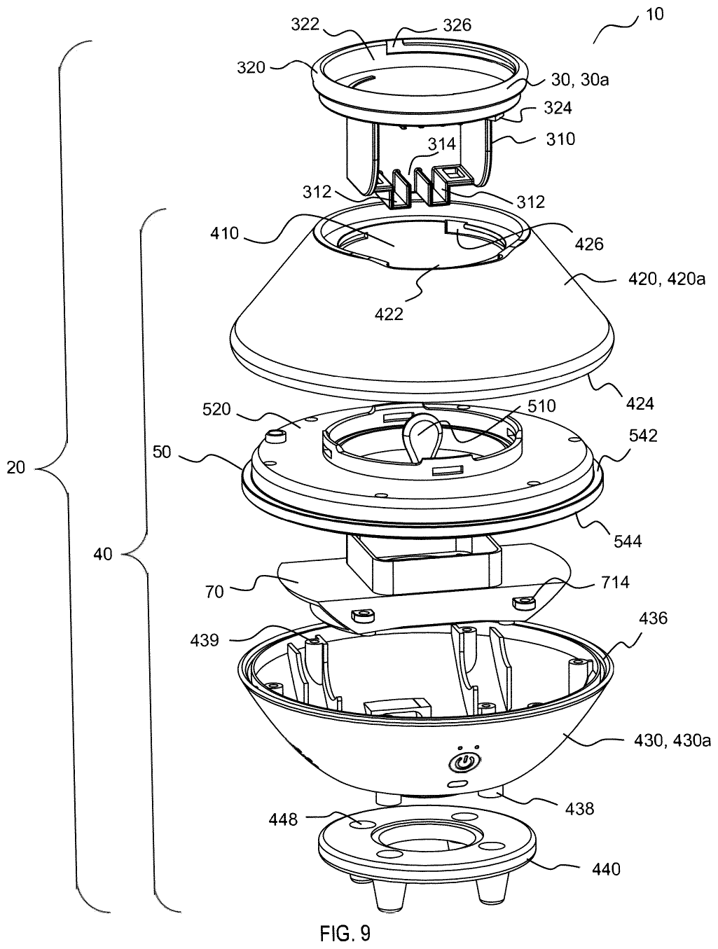

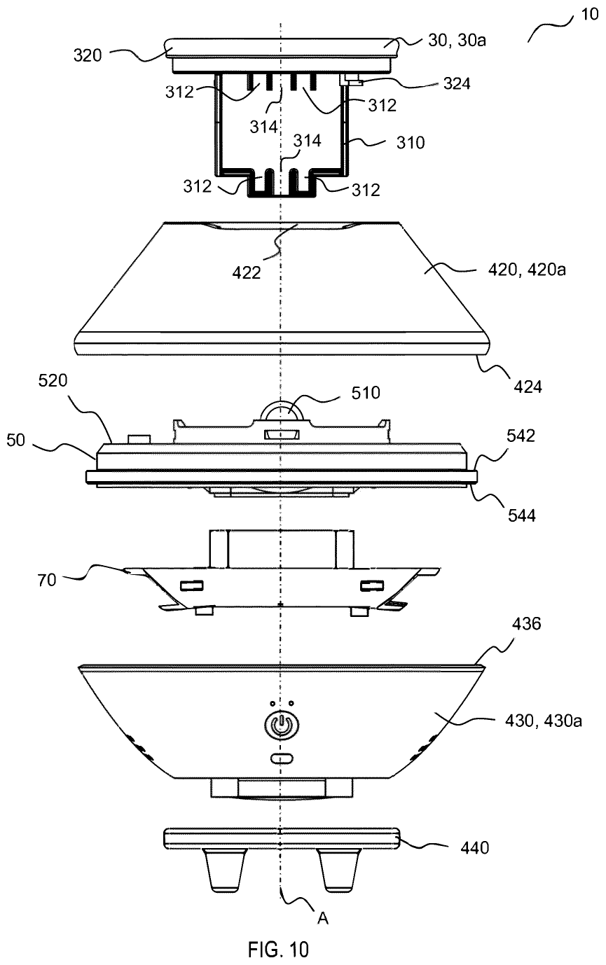

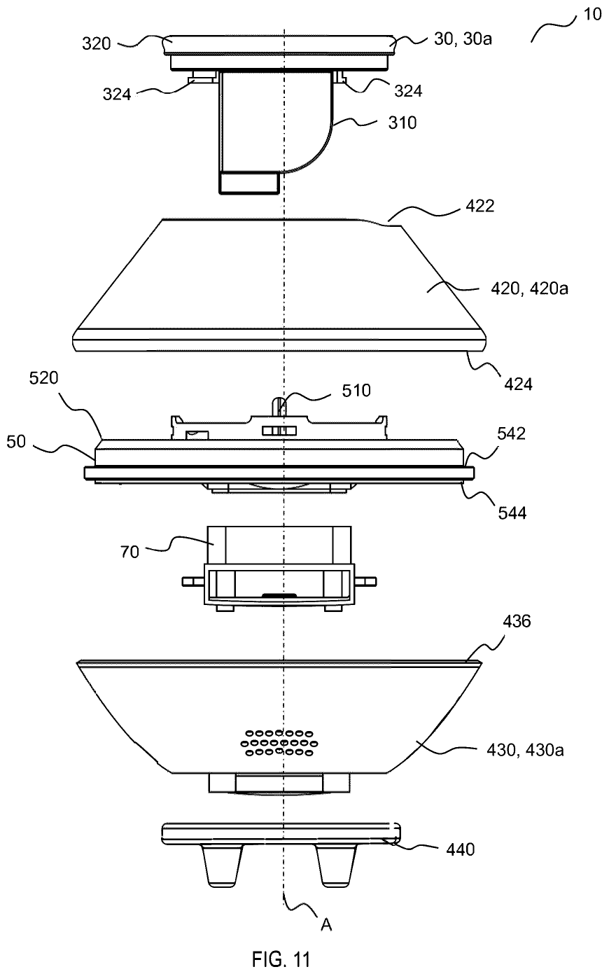

To exemplify and to show the arrangement and interrelationships of various elements of the volatile composition dispenser 10, FIG. 9 provides an exploded upper front perspective view of a volatile composition dispenser 10, FIG. 10 provides an exploded front view, and FIG. 11 provides an exploded left side view of a housing 20 of a volatile composition dispenser 10. Referring to FIGS. 9-10, the volatile composition dispenser 10 may comprise a top portion 30, an upper part 420 of a base portion 40, a rupture assembly 50, an electrical support assembly 70, a lower part 430 of a base portion 40, and a pedestal 440 aligned along a vertical alignment axis A (shown in FIGS. 10 and 11). Details of each component are described in greater detail hereinafter. The base portion 40 may define a cavity 410 into which the top portion 30, such as a refill system carrier 30a, may be inserted. The top portion 30 may comprise one or more alignment members 324, which may align with and engage with alignment members 426 of the upper part 420 of the base portion 40, for example in a releasably interlocking relationship. The upper part 420 of the base portion 40 may also comprise one or more engagement members 424 to engage the lower part 430 and/or the rupture assembly 50. Similarly, the lower part 430 of the base portion may comprise one or more engagement members 436 to engage the upper part 420 and/or the rupture assembly 50. The rupture assembly 50 may comprise one or more top engagement members 542 to engage the engagement members 424 of upper part 420 and one or more bottom engagement members 544 to engage the engagement members 436 of the lower part 430. The electrical support assembly 70 may be disposed within the cavity 410 and may comprise mounting tabs 714 to engage internal mounting members 439. Finally, the lower part 430 of the base 40 may comprise lower engagement members 438 to engage base engaging members 448 of the pedestal 440. Again, for the sake of brevity, FIGS. 9-10 focus on an overview of the arrangement and interrelationships of various elements of the volatile composition dispenser 10 and descriptions of additional details of each component are reserved for later discussion although some of these additional details are labeled in FIGS. 9-10 for reference.

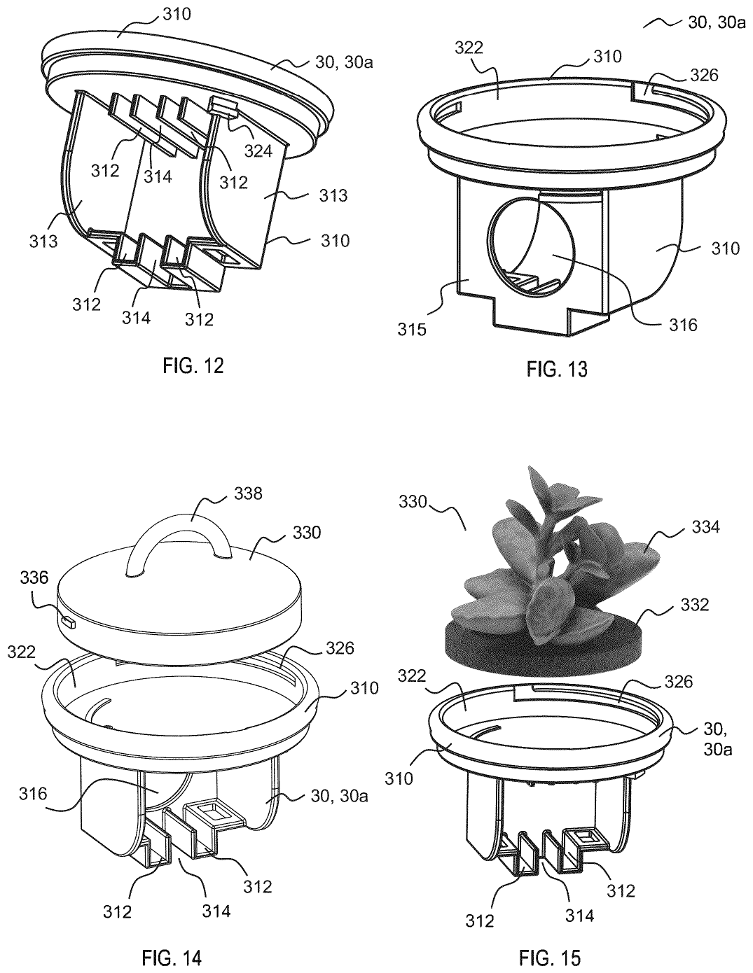

FIG. 12 provides a lower front perspective view, FIG. 13 provides an upper back perspective view, FIG. 14 provides an upper front perspective view, and FIG. 15 provides an upper front perspective view of a top portion 30 of a volatile composition dispenser 10. The top portion 30 may be a refill system carrier 30a, which may comprise an upper part 320 and a lower part 310. The lower part 310 may be inserted into the cavity 410 of a base portion 40. The upper part 320 may interlock with the base portion via one or more alignment members 324. The upper part 320 may comprise a top recess 322, which may comprise removable cap engagement channels 326 for engaging one ore alignment members 336 of a removable cap 330. The removable cap 330 may take a variety of forms. As shown in FIG. 14, the removable cap 330 may comprise a handle 338. As shown in FIG. 15, the removable cap 330 may comprise an artificial plant 334 and an artificial plant substrate 332. The lower part 320 of the refill system carrier 30a may comprise slots 312 for receiving one or more cartridges 610. The slots 312 may be separated by a spacer or a gap 314, which may be sized to accommodate a rupture member 510 to be inserted therethrough. The lower part 320 may comprise side walls 313 and a rear wall 315. An optional aperture 316 may be provided in the rear wall 315. The side walls 313 may help to keep the cartridges locked in place and to keep pressure on from the rupture member 510. The rear wall 315 may stop the cartridges 610 from being pushed too far into the cartridge holder and may keep the cartridges 610 ideally centered about the spindle/rupture member.

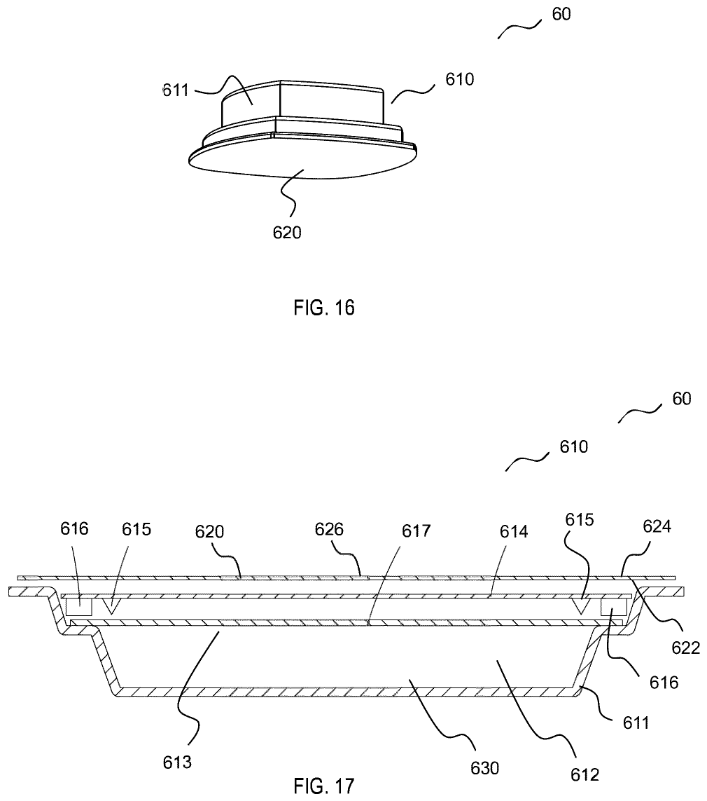

FIG. 16 provides a perspective view, and FIG. 17 provides a cross-sectional side view of a volatile composition cartridge 610. A refill system 60 may comprise one or more volatile composition cartridges 610. The volatile composition cartridge 610 may be configured in a variety of ways. Each cartridge 610 may comprise a body 611 defining a reservoir 612 accessible via an orifice 613. The orifice 613 may be covered or sealed by a rupturable substrate 617 to contain a volatile composition 630 within the reservoir 612. As will be discussed in greater detail hereinafter, the volatile composition may comprise at least 20% of perfume raw materials. A rupture element 614 may be positioned above the rupturable substrate 617. The rupture element 614 may comprise one or more piercing elements 615 supported on a corresponding spring-like part 616. The spring-like part 616 may comprise a metal coil, a polyolefin or polyurethane foam, injection molded bristles, injection molded plastic spring or hinge parts, or the like. Upon pressing the rupture element 614 towards the rupturable substrate 617, for example, by actuating a rupture member 510 (described hereinafter) positioned adjacent to the refill system 60, one or more piercing elements 615 may puncture the rupturable substrate 617. Thereafter, the rupture element 614 may return to its original position by action of the spring-like part 616.

The rupturable substrate 617 may be made of any material that ruptures with applied force, with or without the presence of an element to aid in such rupture. Because the rupturable substrate 617 may contain a volatile composition 630 while in storage, it may be made from any barrier material that prevents evaporation of the volatile composition 630 prior to its intended use. Such materials may be impermeable to vapors and liquids. Suitable barrier materials for the rupturable substrate 617 may include a flexible film, such as a polymeric film, a flexible foil, or a composite material such as foil/polymeric film laminate. Suitable flexible foils include a metal foil such as a foil comprised of a nitrocellulose protective lacquer, a 20 micron aluminum foil, a polyurethane primer, and 15 g/m2 polyethylene coating (Lidfoil 118-0092), available from Alcan Packaging. Suitable polymeric films include polyethylene terephtalate (PET) films, acrylonitrile copolymer barrier films such as those sold under the tradename Barex® by INOES, ethylene vinyl alcohol, and combinations thereof. It is also contemplated that coated barrier films may be utilized as a rupturable substrate. Such coated barrier films include metallized PET, metalized polypropylene, silica or alumina coated film may be used. Any barrier material, whether coated or uncoated, may be used alone and or in combination with other barrier materials.

The internal components of the volatile composition cartridge 610 may be characterized as follows. For example, dimensions of the reservoir 612 may be configured to hold about 1 ml to about 50 ml of the volatile composition 630, preferably liquid. Alternatively, the reservoir 612 may hold about 2 ml to about 30 ml, alternatively about 2 ml to about 10 ml, alternatively about 2 ml to about 8 ml, alternatively about 4 ml to about 6 ml, alternatively about 2 ml, alternatively about 6 ml of a liquid volatile composition 630. Further, a shape of the cartridge 610 may be configured to correspond to a shape of the one or more slots 312 of the top portion 30. For example, the cartridge 610 may define a substantially round, elliptical, or oval shape and its width to length ratio may be about 1:2 to 1:2.5.

A membrane 620 may be attached to the body 611 over the rupture element 614. The membrane 620 may be a composite microporous membrane. The membrane 620 may comprise first side 622 facing the reservoir 612 and a second side 624 opposite the first side 622. The second side 624 may comprise or be coated with a diffusion regulating coating 626, which may comprise a hydrophobic/oleophobic material comprising at least one fluoroalkyl group. The membrane 620 or a portion thereof, such as the diffusion regulating coating 626 may allow release the volatile composition 630.

The membrane 620 may be a microporous membrane 620 having an average pore size of about 0.01 to about 0.06 microns, alternatively from about 0.01 to about 0.05 microns, alternatively about 0.01 to about 0.04 microns, alternatively about 0.01 to about 0.03 microns, alternatively about 0.02 to about 0.04 microns, alternatively about 0.02 microns. Further, the membrane 620 may be filled with any suitable filler and plasticizer known in the art. Fillers may include finely divided silica, clays, zeolites, carbonates, charcoals, and mixtures thereof. The microporous membrane 620 may be filled with about 50% to about 80%, by total weight, of silica, alternatively about 60% to about 80%, alternatively about 70% to about 80%, alternatively about 70% to about 75%. A thickness of the membrane 620 may be about 0.01 mm to about 1 mm, alternatively between about 0.1 mm to 0.4 mm, alternatively about 0.15 mm to about 0.35 mm, alternatively about 0.25 mm.

Still further, an evaporative surface area of the membrane 620 may be about 2 cm² to about 100 cm², alternatively about 2 cm² to about 25 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm². Accordingly, the cartridge body 611 may be sized and shaped to fit the evaporative surface area of the membrane 620.

Suitable membranes 620 for the present disclosure include a microporous, ultra-high molecular weight polyethylene (UHMWPE) optionally filled with silica as described in U.S. Pat. No. 7,498,369. Such UHMWPE microporous membranes 620 include Daramic™ V5, available from Daramic, Solupor®, available from DSM (Netherlands), and Teslin™, available from PPG Industries, and combinations thereof, and membranes 620 available from Microporous LLC.

A volatile material or composition 630 suitable for use in the volatile composition cartridge 610 for a volatile composition dispenser 10 may be configured to condition, modify, or otherwise change the atmosphere and may include compositions suitable for the purposes of providing fragrances, air fresheners, deodorizers, odor eliminators, malodor counteractants, insecticides, insect repellants, medicinal substances, disinfectants, sanitizers, mood enhancers, and aromatherapy aids. A list of the suitable volatile materials is shown in Table 1 below.

TABLE 1

| Purpose | Volatile Material |
|---|---|
| Providing fragrances | Perfume oil, volatile essential oils, volatile organic compound, |
| | synthetically or naturally formed materials. |
| | Examples include, but are not limited to: oil of bergamot, bitter orange, |
| | lemon, mandarin, caraway, cedar leaf, clove leaf, cedar wood, geranium, |
| | lavender, orange, origanum, petitgrain, white cedar, patchouli, neroili, |
| | rose absolute, and the like. |
| | Suitable crystalline solids include but are not limited to: vanillin, ethyl |
| | vanillin, coumarin, tonalid, calone, heliotropene, musk xylol, cedrol, |
| | musk ketone benzohenone, raspberry ketone, methyl naphthyl ketone |
| | beta, phenyl ethyl salicylate, veltol, maltol, maple lactone, proeugenol |
| | acetate, evemyl, and the like. |
| Neutralize malodors | Suitable malodor compositions include reactive aldehydes and ionones |

The volatile composition 630 may be formulated such that the volatile composition 630 comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. The volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

As noted, the volatile composition cartridge 610 may contain a liquid volatile composition 630 disposed in a reservoir 612, wherein the liquid volatile composition 630 is evaporated through a membrane 620. The volatile composition 630 may also be in the form of a solid or semi-solid gel or wax. For example, the volatile composition cartridge 610 may be in the form of a solid gel article. The solid article may be molded with a moldable material such as any one of the gel compositions described hereinafter.

The gel composition may be a chemically cross-linked polyol or derivative thereof. Suitable polyols or derivatives thereof may be selected from the group consisting of: polyol, polyester polyol, polyglycerol and mixtures thereof. Polyols, polyester polyols and polyglycerols comprise multiple hydroxyl groups, and are suitable for forming gels having a compact network. In addition, the resultant gel has greater affinity for hydrophobic materials which are less strongly hydrophobic.

Suitable polyols or derivatives thereof can have a molecular weight of from 60 Da to 10000 Da, preferably from 150 Da to 3000 Da, even more preferably from 500 Da to 2000 Da, even more preferably 600 Da to 1300 Da. Longer polyols and derivatives thereof, result in greater flexibility of the gel.

Suitable polyols and derivatives thereof do not comprise terminal hydroxyl groups. Secondary alcohols are particularly suitable. Primary alcohols, having terminal hydroxyl groups, typically result in more linear chains and a more compact network. A combination of primary and secondary alcohols are preferred, since they result in a more desired correlation length.

An average correlation length of less than 8 nm as measured using Small Angle X-Ray Scattering (SAXS) is preferred. However, the gel compositions, described herein, may be formulated to have any desired correlation length.

A gel with more optimal pore size is achieved when secondary alcohols are used. Lightly branched polyols and derivatives thereof, such as poly(diethyleneglycol adipates) result in more flexible gels. Preferred polyols and derivatives thereof have at least 2 hydroxyl groups per molecule, more preferably at least 3 hydroxyl groups per molecule.

A polyol is a compound containing multiple hydroxyl groups. Diol polyols, having two hydroxyl-functional groups, result after cross-linking in linear polymers or more open networks having large pore size. In contrast, hydroxyl-functional monomers with functionality larger than two form more compact gels with smaller pore sizes. Suitable polyols include: ethylene glycol, propylene glycol, 1,4-butanediol, 1,6-hexanediol, pentaerythritol, 1,2,6-hexanetriol, 4,6-di-tert-butylbenzene-1,2,3-triol, propanetriol (glycerol), 1,2,5-hexanetriol, 1,2,4-cyclohexanetriol, 2,5-dimethylhexane-1,2,6-triol, 3-hydroxymethylpentane-1,2,5-triol, 1,3,6-hexanetriol, 1,1,5,5-pentanetretraol, 1,2,5,6-hexanetretraol, 1,2,3,4,5,6-hexanchexol (sorbitol) and mixtures thereof.

Polyester polyols are hydroxyl-containing esters. Suitable polyester polyols may be selected from the group consisting of: aliphatic polyester polyols, aromatic polyester polyols, organic oil based polyester polyols, and mixtures thereof. Organic oil based polyester polyols are preferred. Preferred organic oils are vegetable oils since they typically comprise high levels of unsaturation (C═C bonds) and naturally comprise hydroxyl groups. Suitable polyester polyols include: hexanoic acid, 4-hydroxy-, 1,1′,1″-(1,2,3-propanetriyl) ester; pentanoic acid, 5-amino-4-hydroxy-, 1,1′,1″-(1,2,3-propanetriyl) ester; Polycaprolactone triol; castor oil, hydroxyl sunflower oil (HSO) and mixtures thereof.

Castor oil is particularly suitable. Castor oil (ricinus oil) is a pale yellow and viscous liquid, derived from the bean of the castor plant (Ricinus communis). Castor oil is predominately made up of triglycerides of fatty acids that contain 87-90% of ricinoleic acid (cis-12-hydroxyoctadec-9-enoic acid), and may be achieved in high purity grades. Castor oil and its derivatives have been used as polyols for polyurethanes and adhesives. The castor oil may be partially hydrogenated. It has been found that castor oil provides the length of the branches and the position of the hydroxyl groups which is particularly suited for providing a chemically cross-linked gel having a pore size which results in slow release of the hydrophobic material, particularly where the hydrophobic material is a perfume. In addition, the chemically cross-linked gels derived from castor oil show less syneresis of the hydrophobic material from the gel.

Polyglycerols are hydroxy-containing ethers. Polyglycerols are typically obtained by the polymerization of alkylene oxides (such as epoxides). Suitable alkylene oxides include ethylene oxide, propylene oxide, butylene oxide, and mixtures thereof, using chain initiators such as ethylene glycol, propylene glycol, diethyelene glycol, dipropylene glycol, 1,4-butanediol, neopentyl glycol, pentaerythritol, hexanetriol, sorbitol, glycerol, and mixtures thereof. Suitable polyglycerols may be selected from the group consisting of: α,α-diglycerol, α,β-diglycerol, hyperbranched polyglycerol, dendritic polyglycerol, and mixtures thereof. Hyperbranched polyglycerols are aliphatic polyethers with multiple hydroxyl end groups that are obtained from the nonsymmetric polyaddition of glycidol to glycerol resulting in a globular branch-on-branch structure which provides special internal flexibility. Dendritic polyglycerols are a hyperbranched polyglycerol with a well-defined symmetric and spherical three-dimensional structure around a core. Apart from improving gel elasticity, the dendritic structure with sterically shielded core together with the exceptionally high number of functional groups of hyperbranched polyglycerols produces flexible gels with relatively low pore size, which increase the longevity of final composition by reducing the diffusion rate not only as a consequence of physically entrapping the hydrophobic material, but also enhancing H-bonding and Van der Waals interactions. Such polyglycerols may be purchased from Nanopartica GmbH (Germany) and Sigma-Aldrich.

Suitable polyglycerols include: polyethylene glycol, polypropylene glycol, poly(diethylene glycol), poly(dipropylene glycol), poly(1,4-butanediol), poly(neopentyl glycol), poly(1,6-hexanediol), and mixtures thereof. The polyglycerol preferably has from 2 to 50, preferably from 4 to 30 repeat units.

Any suitable cross-linking agent may be used, though cross-linking agents selected from the group consisting of: isocyanates, isothiocynates and mixtures thereof, are preferred. The cross-linking agent may be a linear, branched, or cyclic isocyanate, and mixtures thereof. Cyclic isocyanates and mixtures thereof are preferred. Suitable cyclic isocyanates include heterocyclic isocyanates such as 1,3,5-tris(5-isocyanatopentyl)-1,3,5-triazine-2,4,6(1H,3H,5H)-trione.

Suitable cross-linking agents may be selected from the group consisting of: 1,4-butane diisocyanate (BDI), 1,6 hexamethylene diisocyanate (HMDI), L-Lysine ethyl ester diisocyanate (LDI), 4,4′-Methylenebis(cyclohexyl isocyanate) (H12MDI), Glycolide-ethylene glycol-glycolide isocyanate (Bezwada, LLC), 4,4′-Methylenebis(phenyl isocyanate) (MDI), 2,4′-Methylenebis(phenyl isocyanate) (MDI), 2,2′-Methylenebis(phenyl isocyanate) (MDI), Isophorone diisocyanate (IPDI), 2,4-toluene diisocyanate (2,4-TDI), 2,6-toluene diisocyanate (2,6-TDI), Poly (hexamethylene diisocyanate) (PDI), 1,3-bis(2-isocyanatopropan-2-yl)benzene, Poly (pentamethylene diisocyanate) and mixtures thereof, preferably 1,6 hexamethylene diisocyanate (HMDI), L-Lysine ethyl ester diisocyanate (LDI), Poly (pentamethylene diisocyanate), Poly (hexamethylene diisocyanate) (PDI), 1,3,5-tris(5-isocyanatopentyl)-1,3,5-triazine-2,4,6(1H,3H,5H)-trione, and mixtures thereof. Such cross-linking agents are available from Sigma-Aldrich and from Covestro under trade name of Desmodur® eco N 7300.

The cross-linking agent can have a viscosity below 2.500 mPa·s at 25° C. and an isocyanate equivalent weight of from 15% to 40%, preferably from 18% to 30%. Such cross linking agents are more easily blended with the polyol. As a result, more uniform gels may be achieved.

The gel may be formed using a molar ratio of polyol (or derivative thereof) to cross-linking agent of from 1:0.75 to 1:2, preferably from 1:0.8 to 1:1.6, more preferably from 1:0.8 to 1:1.2. Such ratios of polyol to cross-linking agent typically result in gels having an elastic modulus G′ which is of the same order as the viscous modulus G″. In addition, ratios of polyol to cross-linking agent typically result in gels having an elastic modulus G′ of above 0.1 kPa, preferably above 1 kPa, even more preferably above 2 kPa, and below 100 kPa. The gel is preferably essentially free, or free of unreacted isocyanates and/or isothiocyanates.

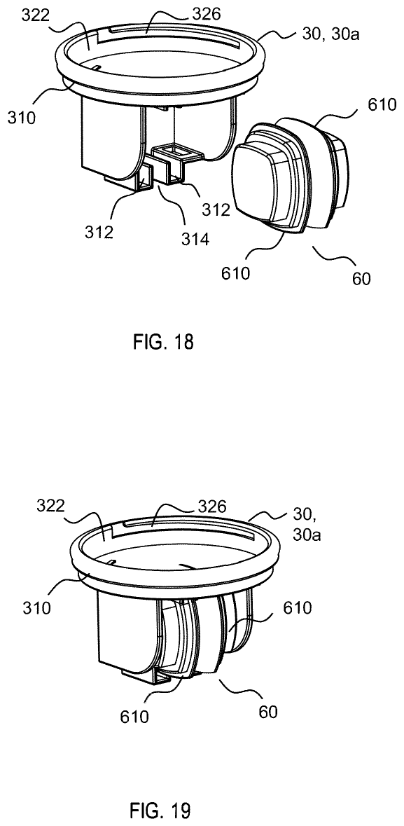

FIG. 18 and FIG. 19 cooperate to illustrate the function of refill system carrier 30a and a refill system 60. FIG. 18 provides a perspective view of a refill system carrier 30a and a disengaged refill system 60. FIG. 19 provides a perspective view of a refill system carrier 30a and an engaged refill system 60. The membranes 620 of two cartridges 610 may be positioned in a facing relationship and the cartridges may be inserted into the refill system carrier 30a to engage with and to be supported by the slots 312.

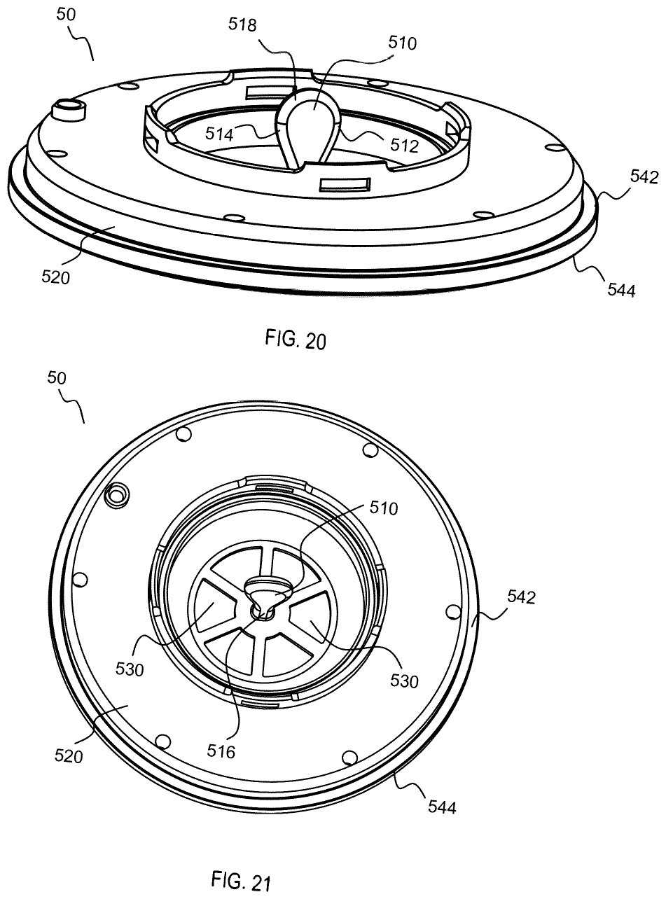

FIG. 20 provides a perspective view of a rupture assembly 50, and FIG. 21 provides a top perspective view of a rupture assembly 50. The rupture assembly may comprise a rupture member 510. The rupture member 510 may comprise a first rupture surface 512 and a second rupture surface 514 opposite the first rupture surface 512. The first rupture surface 512 and the second rupture surface 514 may be disposed on opposite sides of a central spindle 516. The first rupture surface 512 and the second rupture surface 514 may have rounded edges 518 to ensure smooth, non-piercing engagement with the membranes 620 of the cartridges 610 of a refill system 60. The rupture assembly 50 may further comprise a rupture member mount 520 upon which the rupture member 510 is disposed. The rupture member mount 520 may comprise an air inlet 530 comprise one or more apertures to allow air to flow from the lower part 430 of the base portion 40, through the rupture assembly 50, into the upper part 420 of the base portion 40, to contact one or more of the cartridges 610 of a refill system 60 held in a refill system carrier 30a. The rupture member 510 may be injection, compression, or pressure molded using a polyolefin, such as polyethylene or polypropylene; polyester; or other plastics known to be suitable for molding. The rupture member 510 may also be made by thermoforming with a discrete cutting step to remove parts not wanted. The rupture member 510 may additionally or alternatively comprise ceramics or metallics, such as, for example steel, titanium, or aluminum metals, alloys, or composites.

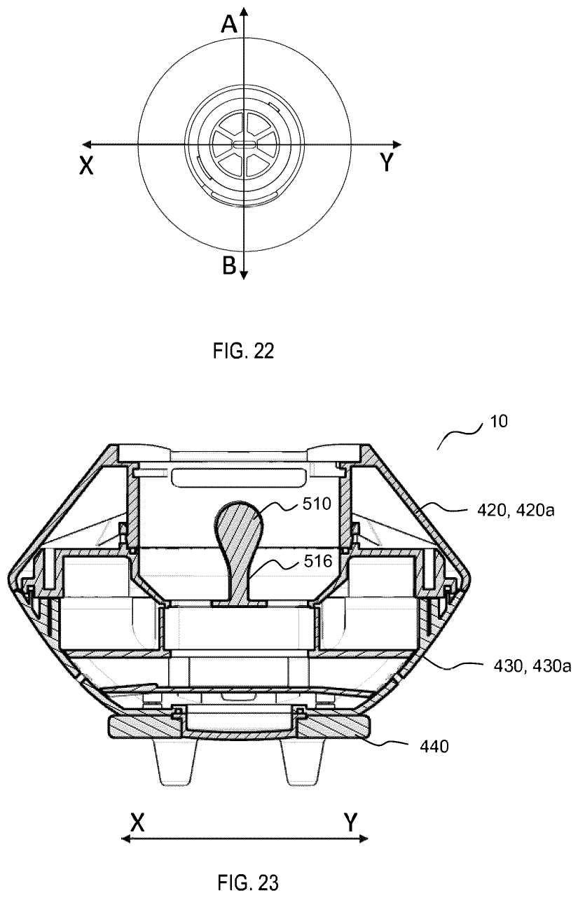

FIG. 22 and FIG. 23 cooperate to illustrate the orientation of a rupture assembly 50 comprising a rupture member 510 within a base portion 40 of a volatile composition dispenser 10. FIG. 22 provides a top view of a base portion 40, and FIG. 23 provides a cross-sectional view along line X-Y of the base portion 40 shown in FIG. 22.

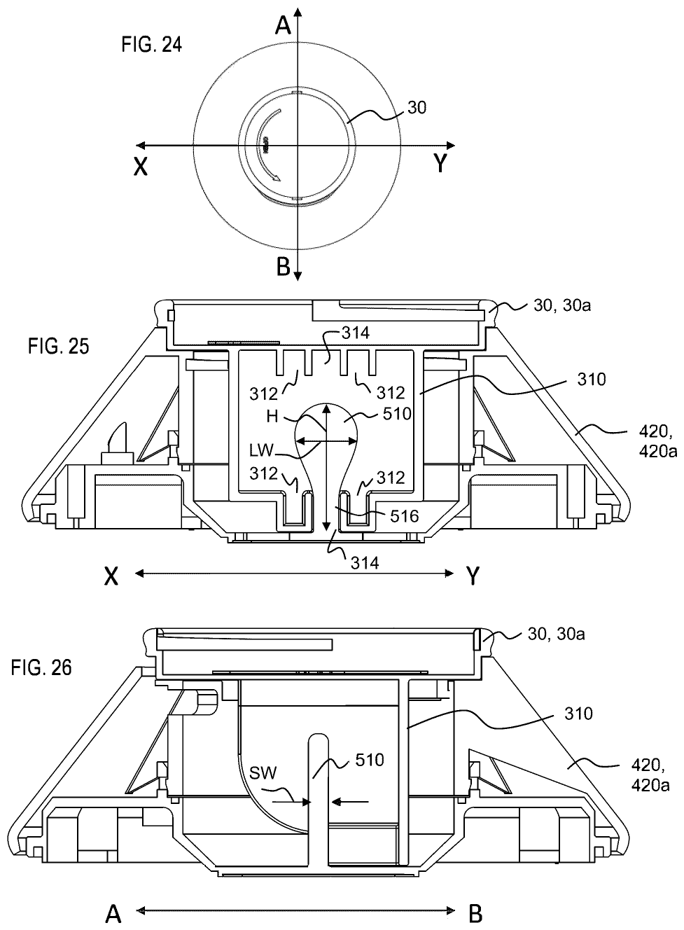

FIG. 24, FIG. 25, and FIG. 26 cooperate to illustrate the orientation of a rupture assembly 50 comprising a rupture member 510 and a refill system carrier 30a within a base portion 40 of a volatile composition dispenser 10. FIG. 24 provides a top view of a housing 20 of a volatile composition dispenser 10, FIG. 25 provides a cross-sectional view along line X-Y of the housing 20, and FIG. 26 provides a cross-sectional view along line A-B of the housing 20. The rupture member 510 is shown generally as a ‘lollipop’ shape, but other shapes are possible and will be readily apparent to those having ordinary skill in the art based upon this disclosure. The body of the rupture member 510 may have a short-axis width SW of 4 mm, or of about 3 mm to about 6 mm. The rupture member 510 may have a long axis width LW of 11 mm, or of about 8 mm to about 15 mm. The height H of the rupture member 510 may be 26 mm, or about 20 mm to 30 mm.

The edges of the rupture member 510 that contact the cartridges 610 of the refill system 60 may be rounded (not flat, no sharp corners) so that, upon rotation, they apply a gradual pressure to the membrane 620 and rupture elements of the refill system 60. The rupture member 510 does not rupture the entire microporous membrane 620, to ensure that the volatile scent must permeate the microporous membrane 620 for scent to be delivered to the air. It will be appreciated that this mechanism can activate a refill system 60 comprising two cartridges 610 simultaneously. By using two cartridges 610, a larger surface area for scent emission can be achieved while still fitting the total reservoir capacity and membrane area within the device 10. Also, no moving appendages are needed to activate the refill system 60 such as a lever or button. This allows for better air-flow and less material taking up interior space in the device 10.

Variations on the rupture member 510 are possible. For example, a three dimensional spindle having three protrusions may be employed to activate cartridges in sequence by rotation through 90, 180, 270 and then 360 degrees, and then lock in place. According to other embodiments a rupture member 510 may be provided with only one protrusion, which may be used to activate cartridges 610 of a refill system 60 in series so that a consumer can activate one cartridge after a 90 degree turn, then later (when first is expired) activate a second cartridge with an additional 90 degree turn (total 180 degree of turn). It will be appreciated that the refill system 60 need not be limited to two cartridges 610. The refill system 60 may comprise a plurality of cartridges 610. For example, a refill system may comprise three cartridges 610, which may be activated simultaneously or in sequence.

The rupture assembly 510 may be equipped with a light and/or heating element. Heat transfer will increase an evaporation rate of the volatile composition. The light and/or heating element may be controlled such that it is only activated when the motion detector 726 has activated the device 10.

End-of-life monitors may be incorporated into the rupture assembly to detect when the refill system needs to be replaced and to provide an indication to the user to change the refill system. For such a purpose a non-contact monitor comprising a light and a sensor may be employed.

The shape of the rupture assembly 510 may optimize air flow across the membranes 620, to optimize and/or to direct air flow to the air outlet 422. In embodiments in which the refill system 60 comprises a plurality of cartridges 610 with different compositions, the rupture assembly 510 may be adapted to automate rotation of rupture assembly 510 so different volatile compositions are delivered at different times.

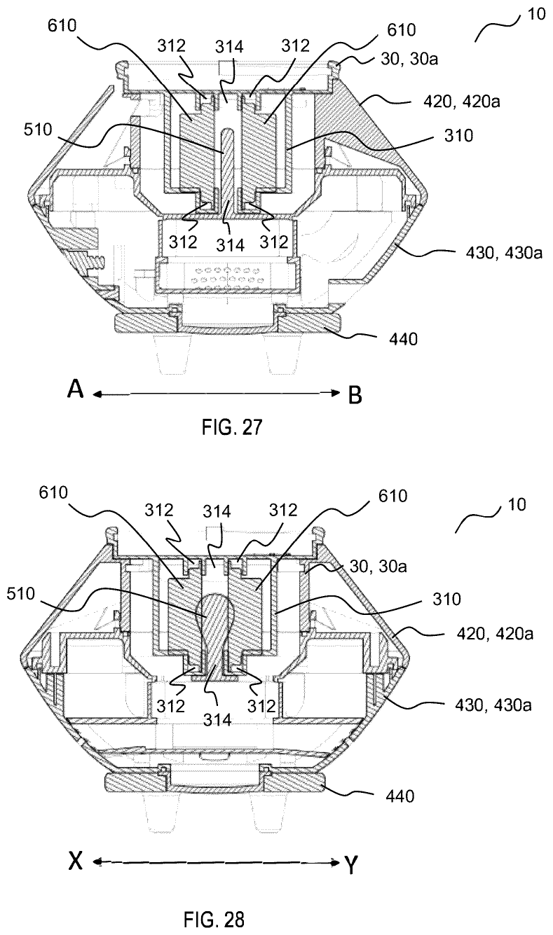

FIG. 24, FIG. 27, and FIG. 28 cooperate to illustrate the orientation of a rupture assembly 50, a refill system carrier 30a, and a refill system 60, within a base portion 40 of a volatile composition dispenser 10. FIG. 24 provides a top view of a housing 20 of a volatile composition dispenser 10, FIG. 27 provides a cross-sectional view along line A-B, FIG. 28 provides a cross-sectional view along line X-Y. As shown in FIG. 27, when the refill system carrier 30a is initially inserted into the cavity 410 of the base 40, the rupture member 510 slides through the gap 314 between the slots 312 and does not contact the membranes 620 of the cartridges 610 of the refill system 60. FIG. 28 shows that after the refill system carrier 30a has been rotated within the cavity 410 by 90 degrees, the first rupture surface 512 and the second rupture surface 514 of the rupture member 510 contact the membranes 620 of the cartridges 610 of the refill system to rupture at least the diffusion regulating coating 626 to allow the volatile composition 630 to be released from the reservoir 612 of each cartridge 610. A refill system carrier 30a containing the refill systems 60, which may comprise one or more cartridges 610, may be placed into the base portion 40 of the volatile composition dispenser 10 with the long axis of the spindle 516 of the rupture member 510 parallel to the microporous membranes 620 of the refill system 60.

Various embodiments provide a mechanism built into an air freshener device, such as a volatile composition dispenser 10 whereby a refill system 60, which may comprise one or more cartridges 610 are activated by applying a rotational pressure to microporous membranes 620 and rupturable films or coatings 626 on one face 624 of the cartridges 610. The rotational pressure presses on rupture elements within the cartridge 610 and against the rupturable film 626, rupturing the film 626, and thus allowing a volatile composition 630 to flow out of a reservoir 612 and contact the microporous membrane 620. According to some embodiments, the refill system 60 comprises two cartridges 610 which are oriented in parallel, and with the microporous membranes 620 facing each other. The two cartridges 610, with the microporous membranes 620 facing each other, are slotted into a refill system carrier 30a with parallel stabilizing slots 312 on the top and bottom for the cartridges 610. The slots 312 of the refill system carrier 30a may maintain the cartridges 610 with a space between the microporous membranes 620 of about 9 mm, or of about 5 mm to 15 mm, or of about 6 mm to about 10 mm.

The refill system carrier 30a, and refill system 60, may be rotated 90 degrees around the rupture member 510. With that rotation, the curved/bulbous protrusions of the first rupture surface 512 and the second rupture surface 514 of the rupture member 510 apply pressure to the refill system 60, thus rupturing at least a portion of the membrane 620, such as the diffusion regulating coating 626, releasing the volatile composition 630. Once the refill system carrier 30a, and thus the refill system 60, has been rotated around the rupture member 510, the pressure applied to the refill system 60 from the rupture member 510 also acts to hold the one or more cartridges 610 of the refill system 60 in place, so if the location of the device 10 is moved the cartridges 610 do not move out of the direction of air flow F from the fan 710, which is positioned under the housing 20.

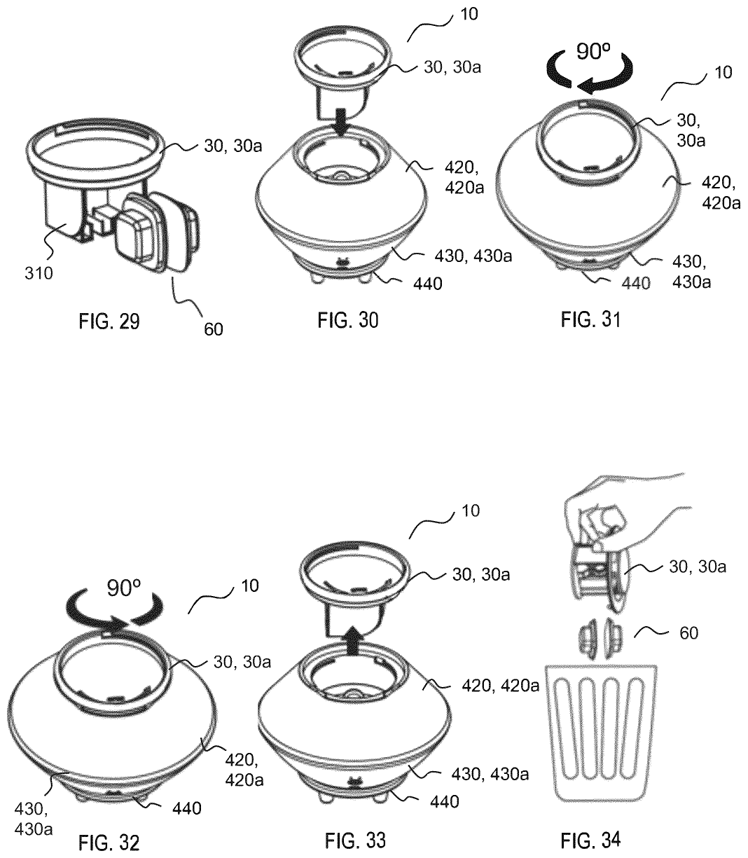

FIG. 29, FIG. 30, FIG. 31, FIG. 32, FIG. 33, and FIG. 34 cooperate to illustrate the interoperation of various components of a volatile composition dispenser 10 as well as a method of using a volatile composition dispenser 10. In general, when the volatile composition cartridge 610 is depleted of volatile composition 630, a user can separate the base portion 40 and the top portion, which may be a refill system carrier 30a, by rotating the base portion 40 and/or the top portion, which may be a refill system carrier 30a, away from each other such. FIG. 29 provides a perspective view of a refill system 60 being inserted into a refill system carrier 30a in a first step. FIG. 30 illustrates a refill system carrier 30a being inserted into a base portion 40 of a volatile composition dispenser 10, in a second step. FIG. 31 illustrates a refill system carrier 30a being rotated within a base portion 40 of a volatile composition dispenser 10 to cause a rupture member 510 to rupture a diffusion regulating coating 626 of a membrane 620, in a third step. Various embodiments provide a highly desirable consumer benefit, allowing for removal and disposal of the refill system once they are expired. The refill system carrier 30a may be rotated 90 degrees in the opposite direction and the holder can be used to dispose of the expired refill systems in the trash without the consumer needing to touch the refill systems or any transferrable compositions associated therewith, such as the volatile composition 630. For example, FIG. 32 illustrates a refill system carrier 30a being rotated within a base portion 40 of a volatile composition dispenser 10 to disengage the refill system carrier 30a from the base portion 40 in a fourth step. FIG. 33 illustrates a refill system carrier 30a being removed from a base portion 40 of a volatile composition dispenser 10, in a fifth step. FIG. 34 illustrates a refill system 60 being released from a refill system carrier 30a in a sixth step.

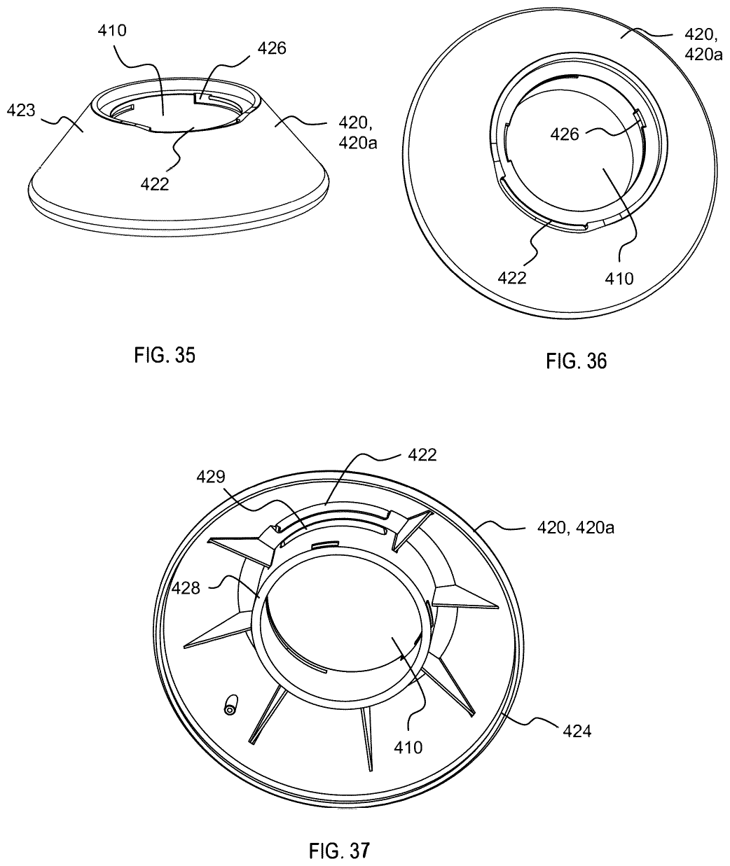

FIG. 35, FIG. 36, and FIG. 37 cooperate to illustrate structural details of an exemplary upper part 420 of a base portion 40 of a housing 20 of a volatile composition dispenser 10. The upper part 420 is illustrated in the form of an upper frustoconical cover 420a. FIG. 35 provides a perspective view, FIG. 36 provides a top perspective view, and FIG. 37 provides a bottom perspective view of an upper part 420, which may be an upper frustoconical cover 420. As best illustrated in FIG. 37, the upper part 420 may comprise internal cavity walls 428, which may surround a portion of the cavity 410 in which the lower part 312 of a top portion 30, such as a refill system carrier 30a, may be inserted. The internal cavity walls 428 may define an internal aperture 429, which may be aligned with and cooperate with the air outlet 422 defined in the side walls 423 of the upper part 420.

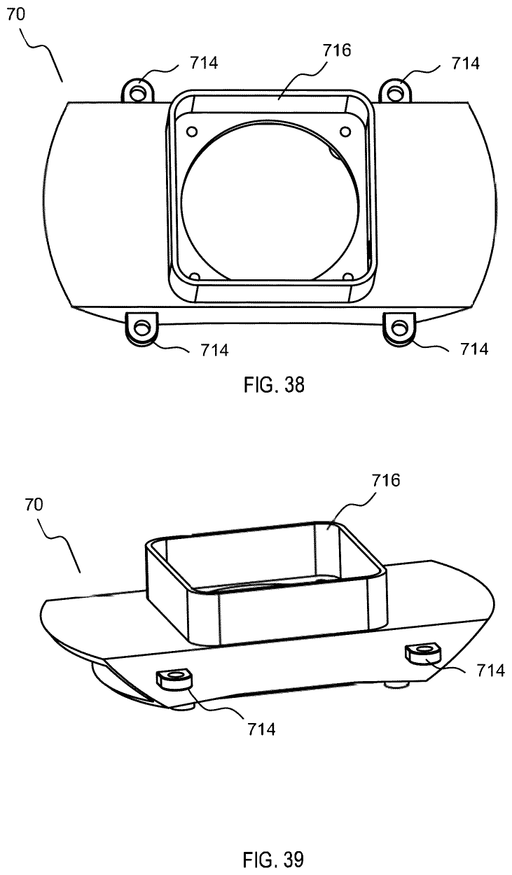

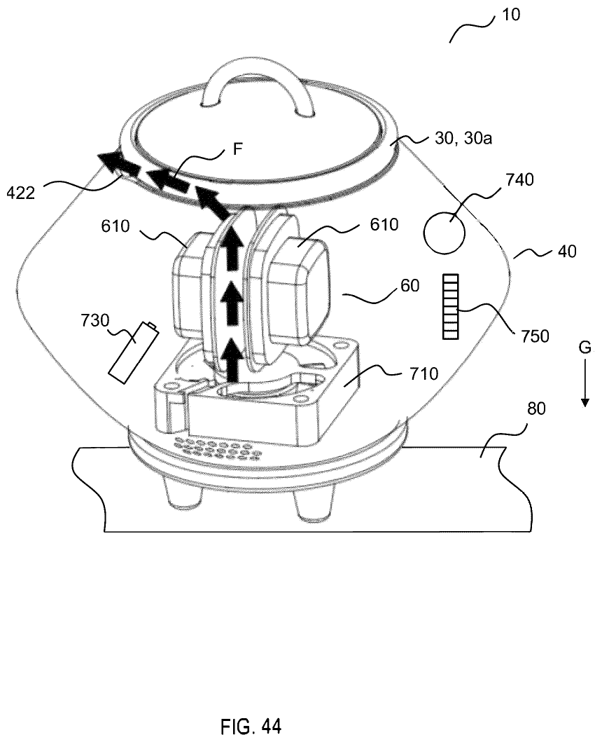

FIG. 38 and FIG. 39 cooperate to illustrate structural details of an electrical support assembly 70 of a volatile composition dispenser 10. FIG. 38 provides a top perspective view, and FIG. 39 provides a perspective view of an electrical support assembly 70 of a volatile composition dispenser 10. The electrical support assembly 70 may support any necessary electrical components, such as for example a fan 712 or one or more batteries 730 (See: FIG. 44), for example via a fan mounting bracket 716. The batteries 730 may be rechargeable and may be charged via a power cable (not shown). The batteries may alternatively be disposable batteries, such as alkaline or nickel cadmium batteries. The batteries may be configured to provide power for a time period of from about 30 to about 45 days per charge. As previously discussed the electrical support assembly 70 may comprise mounting tabs 714 suitable for mounting the assembly 70 within the base 40, such as, for example via internal mounting members 439 of the lower part 430 of the base 40.

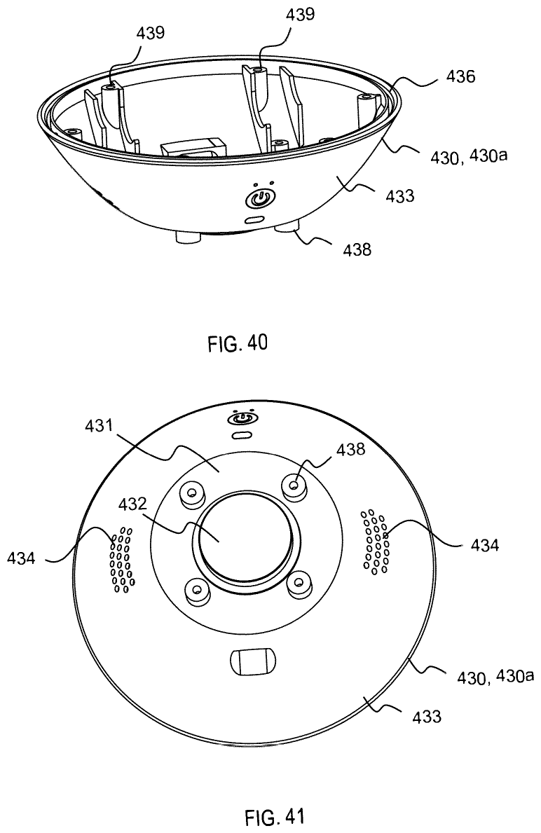

FIG. 40 and FIG. 41 cooperate to illustrate structural details of an exemplary lower part 430 of a base portion 40 of a housing 20 of a volatile composition dispenser 10. The housing 20 may comprise any suitable material, such as, for example, a plastic, a nonflammable material, such as metals, or ceramic. The housing 20 may additionally or alternatively comprise wood or bamboo. The lower part 430 is illustrated in the form of a lower frustoconical cover 430a. FIG. 40 provides an upper front perspective view, and FIG. 41 provides a bottom perspective view of a lower part 430, which may be a lower frustoconical cover 430a of a base portion 40 of a volatile composition dispenser 10. As best illustrated in FIG. 41, the lower part 430 may comprise a bottom surface 431. One or more engagement members 438 may also be disposed on the bottom surface 431 to engage with base engaging members 448 of a pedestal 440.

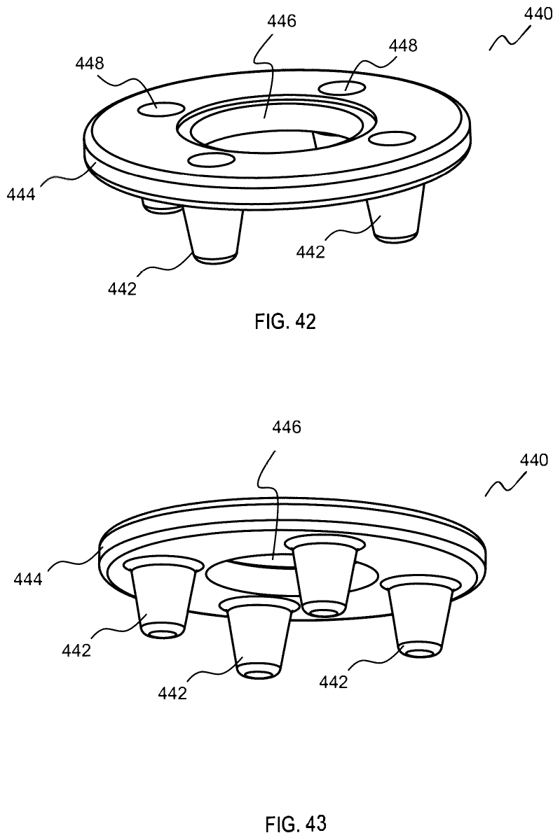

FIG. 42 and FIG. 43 cooperate to illustrate structural details of an exemplary pedestal 440 of a base portion 40 of a volatile composition dispenser 10. FIG. 42 provides an upper perspective view, and FIG. 43 provides a lower perspective view of a pedestal 440 of a base portion 40 of a volatile composition dispenser 10. The pedestal 440 may comprise a base 444 defining an aperture 446, which may allow illumination by an optional light through a translucent lens. The pedestal 440 may also comprise a plurality of legs 442. The pedestal may comprise any suitable material, including, for example, wood, bamboo, ceramic, plastic, or metal.

FIG. 44 is an example according to various embodiments illustrating a schematic diagram of various components of a volatile composition dispenser 10, an airflow F therethrough, and an environment thereof. The volatile composition dispenser 10 may comprise a variety of additional components, such as a night light 740, and/or an intensity control 750, which may regulate the speed of the fan 710 to allow a user to adjust the rate or intensity at which the volatile composition dispersed into the air. As shown, the volatile air dispenser 10 may be positioned on a horizontal surface 80 and may be held in place by the force of gravity G. The fan 712 may provide air that flows through the volatile composition dispenser 10 to deploy any evaporated (i.e., vapor phase) volatile composition from the volatile composition dispenser 10. The fan 712 may be adjustable, for example via the intensity control 750 to vary the amount of volatile composition that is dispensed into the air. An air flow F may proceed through the side air inlets 434 drawn by the fan 710 and may pass through the air inlet 530 of the rupture assembly 50, across the membranes 620 of the refill system 60. Upon passing the membranes 620, the air flow F may entrain a portion of the volatile composition 630. Thereafter, the air flow, comprising the volatile composition 630, may pass through the internal aperture 429 of the upper part 420 (if the internal walls 428 and the internal aperture 428 are present), and finally exit the base 40 through the air outlet 422 to be dispersed into the air surrounding the volatile composition dispenser 10. It may be appreciated that the general direction of the air flow F is in a direction opposite the force of gravity G.

The continuous emission of the at least one volatile composition may be of any suitable length, such as up to 20 days, 30 days, 40 days, 60 days, 90 days, shorter or longer periods, or any period between 10 to 90 days, for example. Of course, more or less volatile composition 630 may be provided in the volatile composition dispenser 10 to increase or decrease its useful life. Also, the volatile composition dispenser's useful life may be dependent on the conditions (i.e., temperature, pressure, moisture content, etc.) in which it operates.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any present disclosure disclosed or claimed herein or that it alone, or in any combination with any other reference or references, teaches, suggests or discloses any such present disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications may be made without departing from the spirit and scope of the present disclosure. It is therefore intended to cover in the appended claims all such changes and modifications that are within the scope of this present disclosure.

What is claimed is:

1. A volatile composition dispenser comprising a housing, the housing comprising:
a base portion and a top portion that is rotatably and releasably connectable with the base portion, wherein the base portion comprises a cavity capable of receiving at least a portion of the top portion, the top portion comprising an upper part and a lower part, wherein:
the lower part comprises one or more slots for receiving one or more volatile composition cartridges,
each of the one or more slots are spaced from one another,
each of the one or more composition cartridges comprise a rupturable substrate,
the base portion comprises a rupture member have a first rupture surface disposed on one side of the rupture member and a second rupture surface disposed on an opposing side of the rupture member,
when the top portion is engaged with the base portion, the lower part is received within the cavity and the rupture member is disposed between the one or more slots and one or more volatile composition cartridges, and
when the top portion is rotated at least 45 degrees, the rupture member engages the rupturable substrate of each of the one or more volatile composition cartridges thereby allowing the volatile composition to be dispensed through the rupturable substrate.

2. The volatile composition dispenser of claim 1, wherein the rupture member is capable of engaging a plurality of volatile composition cartridges simultaneously to allowing the volatile composition to be dispensed from each of the plurality of volatile composition cartridges.

3. The volatile composition dispenser of claim 1, wherein the top portion comprises a recess.

4. The volatile composition dispenser of claim 1, wherein the base portion and/or top portion comprises a fan.

5. The volatile composition dispenser of claim 1, wherein the base portion comprises a fan.

6. The volatile composition dispenser of claim 1, wherein the top portion comprises a fan.

7. The volatile composition dispenser of claim 1, wherein the base portion further comprises legs extending from an outer surface such that a bottom surface of the base portion is elevated above a horizontal surface upon which the base portion sits.

8. The volatile composition dispenser of claim 1, wherein the base portion further comprises an air inlet.

9. The volatile composition dispenser of claim 1, wherein the volatile composition dispenser is powered by batteries or by 120V ac voltage.

10. The volatile composition dispenser of claim 1, wherein the base portion further comprises alignment members which engage with top portion alignment members such that the rupture member does not engage the rupturable substrate upon insertion of the lower part into the cavity.

11. A volatile composition dispenser comprising:
a base portion defining a cavity, the base portion comprising a rupture member disposed within the cavity, the rupture member comprising a spindle and opposing rupture surfaces disposed on and protruding from the spindle;
a refill system carrier comprising one or more slots for receiving one or more cartridges, each cartridge comprising a reservoir sealed via a rupturable substrate,
wherein the slots are spaced to allow the rupture member to slide therebetween without activating the one or more cartridges when the refill system carrier is inserted into the base portion, and
wherein the opposing rupture surfaces are sized to rupture at least a portion of each rupturable substrate simultaneously when the refill system carrier is rotated within the base portion by about 90 degrees.

12. The volatile composition dispenser according to claim 11, wherein the base portion defines an air outlet and an air inlet, and wherein the volatile composition dispenser further comprises a fan positioned within the base portion to move air along a flow path from the air inlet to the air outlet.

13. The volatile composition dispenser according to claim 12, wherein each cartridge comprises a volatile composition sealed within the reservoir by the rupturable substrate, and wherein the flow path intersects with a position of the refill system carrier and the one or more cartridges to entrain the volatile composition in the air.

14. The volatile composition dispenser according to claim 13, further comprising a motion detector configured to activate the fan in response to a detected motion.

15. The volatile composition dispenser according to claim 11, wherein the refill system carrier comprises a top recess.

16. The volatile composition dispenser according to claim 15, wherein the refill system carrier further comprises a removable cap insertable into the top recess.

17. The volatile composition dispenser according to claim 16, wherein the removable cap comprises a handle.

18. The volatile composition dispenser according to claim 16, wherein the removable cap comprises an artificial plant.

19. The volatile composition dispenser according to claim 11, wherein the base portion comprises an upper frustoconical cover and a lower frustoconical cover.

20. The volatile composition dispenser according to claim 19, wherein the rupture member is disposed on a rupture assembly, and wherein the rupture assembly is disposed between the upper frustoconical cover and the lower frustoconical cover.
